(19) United States
(12) Patent Application Publication
(10) Pub. No.: US 2014/0343469 A1
(43) Pub. Date: Nov. 20, 2014

(54) Orthopedic Cast Cover

(71) Applicant: Nellie Bush, Jackson, MI (US)

(72) Inventor: Nellie Bush, Jackson, MI (US)

(21) Appl. No.: 14/268,495

(22) Filed: May 2, 2014

Related U.S. Application Data
(60) Provisional application No. 61/822,960, filed on May 14, 2013.

(51) Int. Cl. A61F 15/00

(52) U.S. Cl. CPC A61F 15/004
USPC 602/1

(57) ABSTRACT

An orthopedic cast cover is provided. The present cast cover provides a removable, sleeve-like device that securely fits over an orthopedic cast to protect skin, clothing, furniture, and other articles from being damaged by the hard, abrasive surface of a plaster or fiberglass orthopedic cast. The present cast cover may further be padded to add an additional layer of protection to the cast cover. The distal end of the cast cover is adapted to fit over the user's hand protruding from the body of the cast and thereby firmly secure it in place. The present invention also has a removable flap or window portion that allows the cast being covered by the present invention to be accessed for activities such as cast signing.

### CROSS REFERENCE TO RELATED APPLICATION

This application claims the benefit of U.S. Provisional Application No. 61/822,960 filed on May 14, 2013 entitled “Cast Cover Clothes Protector.” The above identified patent application is herein incorporated by reference in its entirety to provide continuity of disclosure.

### BACKGROUND OF THE INVENTION

1. Field of the Invention

The present invention relates to orthopedic cast covers. More specifically, the present invention relates to orthopedic cast covers that provide an outer protective layer around the orthopedic cast to prevent the hard, abrasive surface of the cast from damaging clothing, furniture, or other objects.

Orthopedic casts come in both plaster and fiberglass varieties, but in either case the cast has a hard, abrasive surface that can damage clothing, furniture, and other objects in the user's environment. Furthermore, casts that have been worn for extended periods of time can become dirty and unattractive because they cannot easily be cleaned. Lastly, it can be very difficult to conceal or otherwise mask a cast when an individual is seeking to dress nicely. Therefore, there has been a need for soft, padded cast covers that can be used to wrap an orthopedic cast and thereby protect skin, clothing, furniture, and other objects from the rough, abrasive surface of an orthopedic cast.

Current technologies generally consist of a cast cover that extends over the cast of the body and additionally covers the user's exposed hand or foot that protrudes from the body of the cast. While these types of devices adequately protect the rough, hard, abrasive surface of the cast from potentially damaging furniture and articles, they do so at the expense of partial use of the individual's hand. The individual may lose dexterity or the ability to grasp and manipulate objects because his or her hand is covered. Furthermore, such coverings may be hot or uncomfortable to wear and only make the individual's cast even more conspicuous, rather than giving the user the option to mask the presence of the cast.

The present invention solves this problem by providing a removable cast cover that covers an entire orthopedic cast, without also covering the user's extremity that protrudes from the cast. This ensures that the user can retain full use of his or her hand, unimpeded by a bag or other covering. The present orthopedic cast cover is further padded in its preferred embodiments to provide an additional layer of protection for objects that could potentially be damaged by the cast surface. Finally, an elastic edge along the proximal end of the cast cover ensures that the cast cover will remain secure against the cast and will not slip off.

2. Description of the Prior Art

Devices have been disclosed in the prior art that relate to cast coverings. These include devices that have been patented and published in patent application publications. These devices generally relate to bags or sleeves that slip over the outside of the orthopedic cast. The following is a list of devices deemed most relevant to the present disclosure, which are herein described for the purposes of highlighting and differentiating the unique aspects of the present invention, and further highlighting the drawbacks existing in the prior art.

One such device is U.S. Pat. No. 4,986,265 to Caponi, which discloses a protective cover for a cast. The Caponi device consists of an elongated waterproof, flexible polymer bag that fits over the cast and has a sealing band to hold the bag around the user's cast. The Caponi device adequately prevents the generally hard and abrasive material of the cast from catching on clothing and other surfaces, but it does so by covering the individual's entire hand in addition to the cast. This design is undesirable because it affects users' ability to pick up and otherwise manipulate objects. The Caponi device therefore cannot be used at all times unless the user is willing to sacrifice his or her dexterity.

Another such device is U.S. Pat. No. 7,066,899 to Baron, which discloses a washable, decorative orthopedic cast cover that fits over the user's cast and protects it from getting dirty and prevents it from damaging clothing or furniture. The Baron device does not have a means for ensuring that the proximal end of the device does not slide down the user's limb, potentially making the device very uncomfortable to wear and potentially ineffective. The present invention includes an elastic edge along its proximal open end, which holds fast against the user's arm and ensures that the orthopedic cast cover will be held in place.

U.S. Pat. No. 8,343,293 to Wood discloses a device to apply an ornamental design to an orthopedic cast comprising a shrink film in a sleeve-like configuration that conforms to the cast when heat is applied. The Wood device and associated method apply a covering to a cast, but the covering is only decorative and does not cover the cast sufficiently, or with the proper material, to protect furniture or clothing from being damaged by the hard, abrasive surface of the cast. The present invention completely covers the cast with an easy to apply tubular, flexible material that protects clothing and furniture from being scratched by the cast.

Another such device is U.S. Pat. No. 8,382,691 to Rice, which discloses an orthopedic cast attachment that integrates with a cast to provide a protective shell for exposed portions of an individual's body that project from a cast, such as fingers from a wrist cast. Rice does nothing to cover the cast itself, however, and thus does not protect clothing or other objects from being scratched or otherwise damaged by the hard, abrasive surface of the orthopedic cast. The present orthopedic cast cover provides a slip-on, soft cover for an orthopedic cast that prevents the cast from damaging clothing and other objects, such as furniture, while simultaneously offering the user more options to customize the appearance of his or her cast.

U.S. Patent Application Publication No. 2003/0191419 to Melin discloses an orthopedic cast cover that covers the cast and the individual's exposed hand or foot protruding from the cast. This device is intended to protect clothing, furniture, walls, and other such objects in the environment from being damaged by the rough surface of the cast. As with Caponi, individuals may not wish to cover their exposed hands or feet along with the cast because such coverings may make one's hands or feet uncomfortable or too hot and the coverings may be unattractive. Additionally, covers that envelop a user's hand may make picking up, grasping, or otherwise manipulating objects more difficult. The present invention provides an orthopedic cast cover that covers only the user's cast, allowing the user the freedom to choose what foot or hand garments, if any, to use.

The present invention provides a tubular, sleeve-like cast covering that can be used to protect skin, clothing, and surrounding furniture or other articles from being damaged by contact with the hard, abrasive surface of a plaster or fiberglass orthopedic cast. The present orthopedic cast cover is secured firmly in place at its proximal end by an elastic edge and at its distal by a region of material that is thinner than the body of the tubular cast cover. The thinner region of material is adapted to be folded over the edge and under the distal end of an orthopedic cast end. The present invention is designed to fit over an individual's extremity and be secured thereto, without covering the user's entire extremity. A window portion composed of a flap is further disposed along the covering to allow access to the cast covered thereunder. It substantially diverges in design elements from the prior art and consequently it is clear that there is a need in the art for an improvement to existing cast covering devices. In this regard the instant invention substantially fulfills these needs.

### SUMMARY OF THE INVENTION

In view of the foregoing disadvantages inherent in the known types of orthopedic cast coverings now present in the prior art, the present invention provides a new orthopedic cast cover wherein the same can be utilized for providing convenience for the user when protecting skin, clothing, or furniture from being damaged by contact with the surface of an orthopedic cast and concealing the cast so that it matches with clothing.

It is therefore an object of the present invention to provide a new and improved orthopedic cast cover device that has all of the advantages of the prior art and none of the disadvantages.

It is another object of the present invention to provide an orthopedic cast cover that protects clothing, skin, furniture, or anything else that may be damaged through contact with the hard, abrasive surface of a cast.

Another object of the present invention is to provide an orthopedic cast cover that does not also cover, or otherwise impede, the function of the user's extremity that protrudes from the cast.

Yet another object of the present invention is to provide an orthopedic cast cover that allows access to the body of the cast through a window or flap portion.

Yet another object of the present invention is to provide an orthopedic cast cover that is firmly secured in place over the orthopedic cast.

Yet another object of the present invention is to provide an orthopedic cast cover that is freely removable.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the present invention.

FIG. 2 shows a perspective view of the distal end of the present invention.

FIG. 3 shows a perspective view of an alternative embodiment of the present invention, as worn by an individual.

### DETAILED DESCRIPTION OF THE INVENTION

Reference is made herein to the attached drawings. Like reference numerals are used throughout the drawings to depict like or similar elements of the orthopedic cast cover. For the purposes of presenting a brief and clear description of the present invention, the preferred embodiment will be discussed as used for covering an arm cast. The figures are intended for representative purposes only and should not be considered to be limiting in any respect.

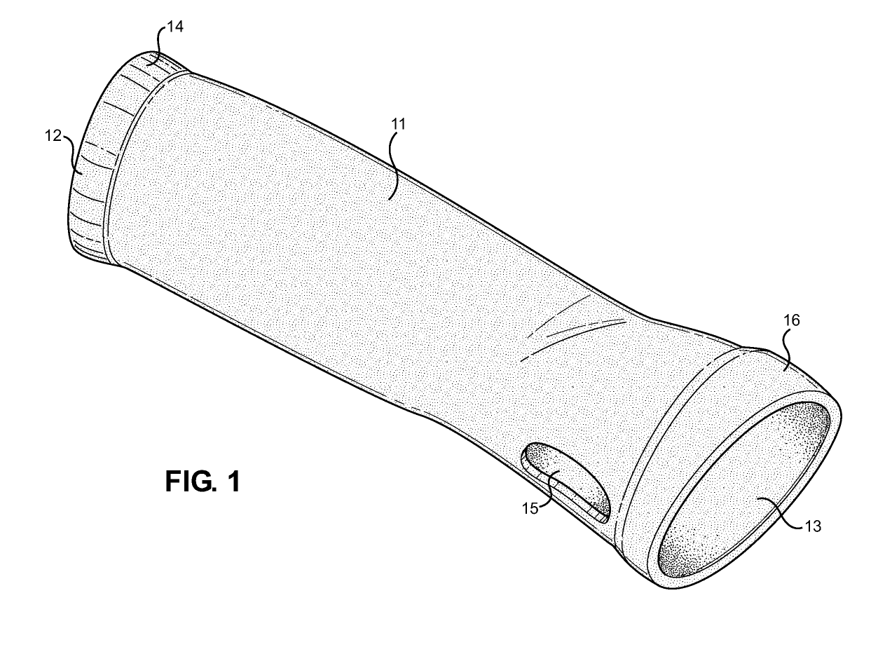

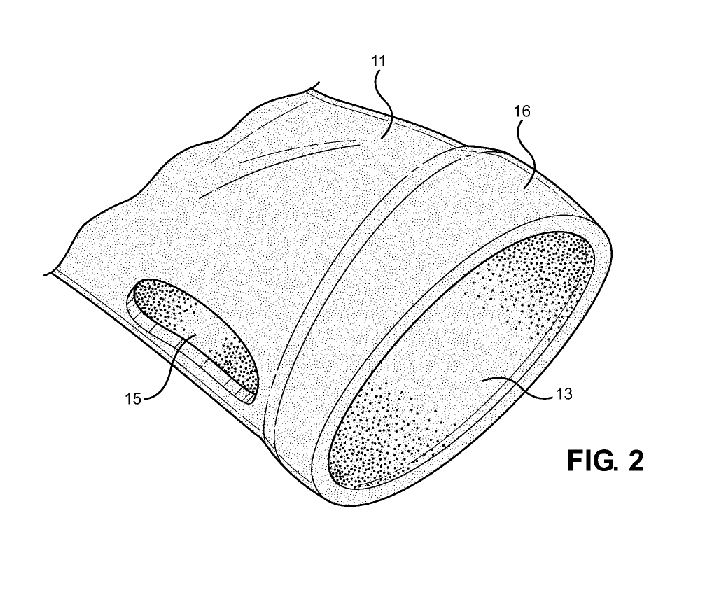

Referring now to FIGS. 1 and 2, there are shown a perspective views of the present orthopedic cast cover. The present invention includes a tubular, sheath-like cover 11 extending between a proximal first open end 12 and a distal second open end 13. The tubular cover portion 11 may be composed of any soft, flexible or elastic material, such as Spandex. The cover 11 material may further comprise a padded layer disposed beneath the outer elastic layer to further serve as a barrier between a cast and surrounding objects. The cover 11 is designed to fit over an orthopedic cast and provide a layer over the hard, abrasive surface inherent to plaster and fiberglass orthopedic casts. The surface of such casts is prone to damaging furniture or other objects, scratching clothing, and irritating skin, so the cover 11 is provided to completely surround the orthopedic cast and provide a non-abrasive, soft layer that dulls the impact of the cast.

In the preferred embodiment, the tubular cover 11 is padded to provide a further barrier between the surface of the cast and outside objects. The padding may be included either as a separate layer beneath the material of the cover 11 or may be integrated into the material of the cover 11 itself. The padding may consist of any material that adequately shields and dulls the impact from objects coming into physical contact with the user's cast, such as cotton, felt, or foam. The padding is substantial enough to provide a protective layer over the cast, but is not so think that it causes the present invention to be bulky or otherwise interfere with the individual's use of their extremity.

An elastic edge 14 is disposed around the perimeter of the generally elliptical open aperture that defines the first open end 12 and is adapted to hold the cover 11 securely in place around the proximal end of the orthopedic cast. The proximal end of the orthopedic cast is defined as the portion of the cast that is higher up on the user's arm when the present invention is secured over a cast. The elastic edge 14 is tight enough to hold the cover 11 in place, but may be stretched so that the user can easily and simply slide the cover 11 on and off his or her cast.

The distal end of the cover 11 has both a second open end 13 and a thumb hole 15. The second open end 13 is a generally elliptical open aperture that is adapted to receive and secure tightly around the user's hand at the end of the orthopedic cast. The second open end 13 is adapted to be secured to the end of the covered orthopedic cast. In one embodiment, the second open end 13 of the cover 11 is removably secured to the distal end of the orthopedic cast by a thin region 16 of material that is thinner than the rest of the body of the cover 11, such that the thinner portion may be tucked underneath the distal end of a cast to secure the second open end 13 of the cover thereto. However, any means known in the prior art may be used to secure the second open end 13 to the distal end of the cast. In the preferred embodiment of the present invention, the thumb hole 15 has reinforced stitching around its perimeter to increase the strength of the present invention and prevent tearing or increased wear on the tubular cover 11.

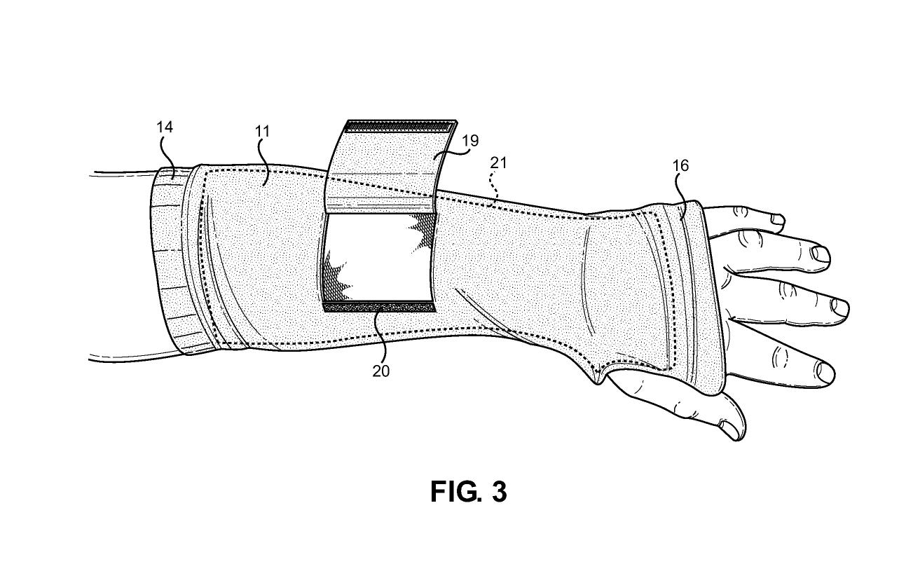

Referring now to FIG. 3, there is shown a perspective view of the present orthopedic cast cover as worn by a user. The cover 11 removably slips over and covers a user's orthopedic cast 21 to provide a protective layer thereover. In the preferred embodiment, as depicted, the present invention further comprises a flap 19, which may be opened to provide access to the cast 21 underneath. When the user wishes to fully cover up the cast 21, the flap 19 may be removably secured in place via a fastener 20, which may comprise complementary strips of hook-and-loop fastener material, buttons, or any other such removable attachment. The flap 19 provides access to the cast 21 thereunder so that individuals can temporarily have access to the cast 21 to sign it, without having to remove the entire cover 11.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) An orthopedic cast cover, comprising:
a tubular cover having a first open end and a second open end;
wherein said tubular cover is composed of a flexible material;
wherein said tubular cover is adapted to fit over and protect an orthopedic cast;
said first open end having an elastic edge adapted to hold said first open end in place along an end of an orthopedic cast;
a thumb hole disposed adjacently to said second open end;
said thumb hole and said second open end adapted to accept a user's hand therethrough.

2) The orthopedic cast cover of claim 1, further comprising a padded layer disposed on said tubular cover.

3) The cast cover of claim 1, further comprising:
a flap disposed on said tubular cover;
said flap adapted to allow access to a cast therethrough;
said flap removably secured to said tubular cover by a fastener.

4) The orthopedic cast cover of claim 3, wherein said fastener comprises hook-and-loop fastener material.

5) The orthopedic cast cover of claim 1, wherein said second open end further comprises a thin region of material that is adapted to be tucked under the distal end of a cast to secure the tubular cover thereto.

6) The orthopedic cast cover of claim 1, further comprising reinforced stitching disposed about the perimeter of said thumb hole.
